(19) United States
(12) Patent Application Publication
(10) Pub. No.: US 2026/0083435 A1
(43) Pub. Date: Mar. 26, 2026

(54) IMPROVED SAW-SENSOR WITH THIN PIEZOELECTRIC LAYER AND OSMOTIC SENSOR DEVICE INCLUDING THE SAME

(71) Applicant: Johann Wolfgang Goethe-Universität Frankfurt am Main, Frankfurt am Main (DE)

(72) Inventor: Michael HUTH, Usingen (DE)

(21) Appl. No.: 19/120,861

(22) PCT Filed: Nov. 15, 2023
(86) PCT No.: PCT/EP2023/081929

§ 371 Date: Apr. 14, 2025

(30) Foreign Application Priority Data
Nov. 15, 2022 (EP) 22207572.3

(51) Int. Cl. A61B 8/00

(52) U.S. Cl. CPC A61B 8/4483; A61B 8/565

(57) ABSTRACT

A SAW sensor comprising: a substrate, a piezoelectric layer deposited on the substrate, an input IDT configured for converting input electric signals into surface acoustic waves, and an output IDT configured for converting said surface acoustic waves into output electric signals, wherein the input IDT and the output IDT are arranged on the piezoelectric layer. TAn osmotic sensor device for measuring a property of a body fluid comprising a cavity for confining a fluid, a membrane for allowing osmotic diffusion into and/or from the cavity, and such SAW sensor. A method of manufacturing such SAW sensor comprising depositing the piezoelectric layer on the substrate by means of a deposition process.

### FIELD

The present invention is in the field of surface acoustic wave sensors. In particular, the invention refers to an improved surface acoustic wave detector usable in an osmotic sensor device for continuously measuring a property of a body fluid such as a glucose concentration in the blood of a patient suffering from diabetes.

### BACKGROUND

In its simplest form, osmosis is a physical process in which a solvent moves across a membrane that is selectively permeable to that solvent and separates two solutions having different concentrations of a solute. The different concentrations cause a net movement of diffusion of the solvent through the membrane from the solution with lower solute concentration to the solution with higher solute concentration, in a trend to equalize the solute concentration on the two sides of the membrane. As a result, a so called “osmotic pressure” builds up in the volume containing the solvent with higher solute concentration, as the concentration difference between the two solutions separated by the membrane decreases. Eventually, a dynamic osmotic equilibrium is reached, in which the increase in chemical potential caused by the osmotic pressure is equalized by a corresponding change in chemical potential caused by the differences in concentration.

Osmosis hence allows inferring the concentration of a solvent based on a pressure measurement. Based on a measurement of the osmotic pressure, a concentration of a solute can be determined. A use of this technique for measuring blood glucose using an osmotic sensor was described in the PHD Thesis “Osmotic sensor for blood glucose monitoring applications”, by Olga Krushinitskaya, Department of Micro-and Nanosystems Technology, Vestfold University College, August 2012.

One known possibility for converting a hydrostatic pressure, the osmotic pressure, into a processable electronic signal is via the piezoelectric effect. The use of piezo-resistive elements for performing measurements of osmotic pressures for determining blood glucose levels is disclosed in WO 2004/107972 A1 and in WO 2018/226104 A1.

A particular type of sensors that use the piezoelectric effect for converting a mechanical measurement of an osmotic pressure into a processable electric signal are the so-called SAW (Surface Acoustic Wave) sensors. Surface acoustic waves are acoustic waves traveling along the surface of an elastic material, which were first explained in 1885 by Lord Rayleigh. SAW sensors typically have a piezoelectric substrate and so-called interdigital transducers (IDT) arranged on the surface of the piezoelectric substrate. When a variable voltage is applied to an input IDT arranged on the piezoelectric substrate, the inverse piezoelectric effect causes the formation of surface acoustic waves that are transmitted along the surface of the substrate and are received by an output IDT also arranged on the piezoelectric substrate, which can transform, due to the piezoelectric effect, the acoustic waves into an output electric signal. A pressure applied to the piezoelectric substrate, which may correspond to the osmotic pressure to be measured, can affect properties of the surface acoustic waves and can hence manifest in the output electric signal. The use of a SAW-sensors for measuring an osmotic pressure is described in EP 2 158 840 A2.

However, SAW sensors known from the prior art typically require the use of a special piezoelectric substrates that makes their integration in most available microelectronic-mechanical systems and osmotic sensors complex and costly.

Therefore, there is room for technical improvement in the field of SAW sensors, in particular when applied to osmotic sensors.

### SUMMARY

One aim of the present invention is providing a SAW sensor that can be manufactured with reduced complexity and reduced costs and can be easily integrated with existing microelectronic-mechanical systems and osmotic sensors, in particular using membrane-like and/or cantilever substrates. Most suitable piezoelectric materials are crystalline or ceramic materials exhibiting high rigidity that are incompatible with the use of this kind of substrate, for which substrates of this type are not being used in known SAW sensors. This, among other technical advantages, is achieved by a SAW sensor.

The SAW sensor of the invention comprises, in particular instead of a conventional piezoelectric substrate, a substrate, preferably a non-piezoelectric substrate, and a piezoelectric layer deposited on the substrate, i.e. arranged on the substrate using a deposition technique, preferably by means of a physical deposition process, such as a sputtering process, or by means of a chemical deposition process, such as atomic layer deposition (ALD), chemical vapor deposition (CVD) or a Langmuir-Blodgett deposition process.

The use of deposition for forming the piezoelectric layer allows that, in some preferred embodiments, a thickness of the piezoelectric layer may be smaller than a thickness of the substrate, preferably 100 to 10000 times smaller, more preferably 500 to 5000 times smaller. This implies that a ratio of a thickness of the piezoelectric layer over a thickness of the substrate may preferably be from 0,0001 to 0,01, more preferably from 0,0002 to 0,002. In some embodiments, a thickness of the piezoelectric layer may be from 0,1 μm to 1 μm, preferably from 0,14 μm to 0,6 μm. In some embodiments, a thickness of the substrate may be from 100 μm to 1000 μm, preferably from 300 μm to 700 μm.

A material composition of the piezoelectric layer may be different from a material composition of the substrate. In some embodiments, the piezoelectric layer may comprise or be made of any of AlN, GaN, InN, ZnO, quartz, LiNbO₃, a piezoelectric polymer or any combination thereof.

The piezoelectric properties necessary for the operation of the SAW sensor are hence provided by a relatively thin piezoelectric layer. Meanwhile, the substrate needs not be a piezoelectric material and may dominantly determine the mechanical structural properties of the SAW sensor. In some embodiments, the substrate may be non-piezoelectric. The substrate may be a silicon substrate. In some preferred embodiments, the substrate may comprise or be made from a flexible and/or elastic material, preferably Si, SiN, SiOx or a combination thereof. By comprising a flexible and/or elastic material, the substrate can adaptively react to changes in a pressure applied thereon, in particular in an osmotic pressure applied thereon, without breaking.

For example, the substrate may comprise a silicon bulk and a layer of SiN formed thereon. The SiN layer may be formed on the silicon bulk by a deposition technique, for example using low-pressure CVD.

The substrate may further comprise an etch stop layer, for example an etch stop layer made of or comprising silicon oxide. The etch stop layer may be arranged between the bulk and the SiN layer or between a bottom portion of the substrate and a bottom portion of the substrate. The etch stop layer may have a thickness from 100 nm to 1000 nm.

According to further examples, the substrate may be a silicon substrate, in particular a substrate made of silicon with (111) crystallographic orientation, which may be beneficial for forming thereon a piezoelectric layer, for example of AlN.

Thus, existing membrane-like or cantilever substrates can be used as a substrate according to the present invention. This allows easily integrating the SAW sensor of the invention with existing microelectronic-mechanical systems and osmotic sensors. A SAW sensor according to the invention may be manufactured with reduced complexity and reduced costs as compared to previously known SAW sensors, for example as compared to the SAW sensors described in EP 2 158 840 A2.

The SAW sensor of the invention further comprises an input interdigital transducer, IDT, configured for converting input electric signals into surface acoustic waves, and an output IDT configured for converting said surface acoustic waves into output electric signals. The input IDT and the output IDT are arranged on the piezoelectric layer. The input electric signals may be wirelessly induced on the input IDT, for example by electromagnetic waves, such as e.g. RF-waves, that may in some examples be received from an external remote control device by an input antenna coupled to the input IDT. Likewise, the output electric signals may be induced in an external monitoring device by electromagnetic waves emitted by an output antenna coupled to the output IDT.

The aforementioned input antenna and/or output antenna may be configured as a single antenna structure usable both as an input antenna and as an output antenna, i.e., may in some embodiments be one and the same antenna device. The antenna structure may be configured to operate in a frequency range from 2.4 GHz (ISM band) to 5 GHz. The antenna structure may be or comprise a loop antenna comprising a loop element and a coupling feed. The loop element may for example be formed as a wire loop with a length corresponding to half the wavelength with which the antenna structure is configured to operate. For example, for an operation frequency of 2.4 GHz, the wire forming the loop element may have a length of about 125 mm and, preferably, a thickness (diameter) of about 1 mm. Each of the coupling feed and the loop element of the antenna structure may be independently connected with the input IDT and/or the output IDT of the SAW sensor of the invention. For example, there may be a first (feed) connection between the IDT device and the coupling feed and a second (grounding) connection between the IDT device and the loop element. The antenna structure may be of reduced size, for example having dimensions 1,6 mm×0,8 mm×0,4 mm. The antenna structure may be or comprise a commercially available dual band antenna.

When an input variable voltage is applied to the input IDT, for example by remote electromagnetic induction via the aforementioned input antenna, the input IDT interacts with the piezoelectric layer, via the inverse piezoelectric effect, generating surface acoustic waves that propagate along the piezoelectric layer. These surface acoustic waves are detected by the output IDT, which interacts with the piezoelectric layer, via the piezoelectric effect, generating an output variable voltage, which may be used to emit electromagnetic waves by means of the aforementioned output antenna.

In some embodiments, the input IDT and the output IDT may be different IDTs, which may be spaced apart from each other. The substrate and/or a portion of the piezoelectric layer may extend between the input IDT and the output IDT. A separation distance between the input IDT and the output IDT may be appropriately chosen to be sufficiently small for avoiding absorption losses, which may otherwise significantly occur if the surface acoustic waves must cover large distances between the input IDT and the output IDT. This separation distance may preferably correspond to 1 to 3 times a wavelength of the surface acoustic waves to be transmitted.

In preferred embodiments, the input IDT and the output IDT may be one and the same IDT device. Thus, one IDT may operate both as the input IDT and as the output IDT. An input variable voltage may be applied to the IDT device, for example by remote electromagnetic induction via an antenna coupled thereto configured to receive input electromagnetic waves. The IDT device, acting as input IDT, may then interact with the piezoelectric layer, via the inverse piezoelectric effect, generating surface acoustic waves that propagate along the piezoelectric layer. These surface acoustic waves may be reflected back to the IDT device, for example by an acoustic reflector, which may for example be or comprise a further IDT device. The reflected surface acoustic waves may then be detected by the IDT device, acting now as the output IDT, which interacts with the piezoelectric layer, via the piezoelectric effect, generating an output variable voltage, which may be used to emit output electromagnetic waves by means of the antenna. Notably, the same antenna may be used for receiving the input electromagnetic waves and to emit the output electromagnetic waves.

According to some embodiments, the SAW sensor may further comprise one or more resonator structures configured for reflecting said surface acoustic waves, generated by the input IDT, to the output IDT, wherein the one or more resonator structures are arranged on the piezoelectric layer. A “resonator structure” may refer herein to any structure able to reflect surface acoustic waves, thereby acting as the previously mentioned acoustic reflector. If the input IDT and the output IDT are the same IDT device, the one or more resonator structures may be configured for reflecting surface acoustic waves generated by the IDT device (when operating as input IDT) back to the IDT device, which then operates as output IDT. The resonator structures may be configured as resonator electrode lattice structures, which for example be made of a conductive material such as Al or Au. Preferably, the one or more resonator structures are arranged on the piezoelectric layer separated from the output IDT and/or from the input IDT.

A separation distance between each of the resonator structures and each of the IDT devices of the SAW sensor (e.g., the input IDT and the output IDT) may be appropriately chosen to be sufficiently small for avoiding absorption losses, which may otherwise significantly occur if the surface acoustic waves must cover large distances between the IDT device(s) and the corresponding resonator structure. This separation distance may preferably correspond to 1 to 3 times a wavelength of the surface acoustic waves to be transmitted.

The one or more resonator structures may be electrically floating structures, i.e. may be electrically isolated from other structures, meaning that the one or more resonators may have no electrical connection with any other element, in particular with no voltage or current source.

Preferably, the one or more resonator structures are arranged on the piezoelectric layer separated from the output IDT and/or from the input IDT. For example, the one or more piezoelectric layers may be separated from the output IDT and/or from the input IDT by a portion of the substrate and/or by a portion of the piezoelectric layer extending between the one or more resonator structures and the input IDT and/or the output IDT.

In preferred embodiments, the one or more resonator structures may comprise at least two resonator structures: a first one of the one or more resonator structures and a second one of the one or more resonator structures. In such embodiments, the input IDT and/or the output IDT may be arranged between the first one of the one or more resonator structures and the second one of the one or more resonator structures. In embodiments in which the input IDT and the output IDT are the same IDT device, the IDT device may be arranged between the first one of the one or more resonator structures and the second one of the one or more resonator structures. This configuration allows an interference of surface acoustic waves reflected by the first resonator with surface acoustic waves reflected by the second resonator to be detected by the output IDT or by the IDT device. A detection of physical properties affecting the piezoelectric surface, for example a pressure applied to the piezoelectric surface, based on such interference may provide a more robust and accurate measurement of said physical properties using the SAW sensor.

In preferred embodiments, the first one of the one or more floating resonator structures and the second one of the one or more floating resonator structures may be identical lattice structures. Additionally or alternatively, the first one of the one or more floating resonator structures and the second one of the one or more floating resonator structures may be symmetrically arranged with respect to the output IDT and/or the input IDT (or with respect to the IDT device that may act both as input IDT and as output IDT). For example, if the IDT device is arranged between the first and second resonators, a distance from the first resonator to the IDT device may equal a distance from the second resonator to the IDT device, with the IDT device being arranged halfway between the first and second resonators and with the first and second resonators being in a symmetric arrangement with respect to the IDT device.

According to preferred embodiments, the output IDT and/or the input IDT are arranged on a first portion of the piezoelectric layer and at least one of the one or more resonator structures are respectively arranged on further portions of the piezoelectric layer, said further portions of the piezoelectric layer being separated from said first portion of the piezoelectric layer, in particular by the substrate. For example, in the previously described embodiments in which the output IDT and/or the input IDT are arranged between a first one of the one or more resonator structures and a second one of the one or more resonator structures, the output IDT and/or the input IDT are arranged on the first portion of the piezoelectric layer, the first one of the one or more resonator structures may be arranged on a second portion of the piezoelectric layer and the second one of the one or more resonator structures may be arranged on a third portion of the piezoelectric layer, wherein each of the second and third portions of the piezoelectric layer may be separated from the first portion.

A further aspect of the invention refers to an osmotic sensor device for measuring a property of a body fluid of a patient, in particular of blood or of interstitial fluid of the patient. The sensor device comprises a cavity for confining therein a fluid and a membrane for allowing osmotic diffusion into and/or from the cavity. A “membrane” as used herein for the osmotic sensor device, may refer to a semipermeable membrane configured for being selectively permeable with respect to a target substance while being impermeable to other substances.

The osmotic sensor device of the invention further comprises a SAW sensor according to any of the previously described embodiments of the first aspect of the invention that is configured for sensing an osmotic pressure in the cavity. For example, the SAW sensor may be arranged such that the substrate thereof is in contact with the cavity, such that an osmotic pressure in the cavity can exert mechanical pressure on the substrate and, via the substrate, also on the piezoelectric layer. As a consequence, the transmission of surface acoustic waves along the piezoelectric layer of the SAW sensor will be affected by the osmotic pressure in the cavity. Consequently, the SAW sensor can sense the osmotic pressure on the cavity.

In preferred embodiments, the cavity may be formed within the substrate of the SAW sensor. The cavity may be enclosed by the membrane and by the substrate and/or the piezoelectric layer of the SAW sensor. In embodiments in which the substrate comprises an etch-stop layer, the cavity may be enclosed by the etch-stop layer.

According to preferred embodiments, a fluid, in particular a liquid, may be confined in the cavity, which fluid may comprise a glucose-binding solute. The cavity may hence be filled with the fluid, which may be a fluid solution able to trap glucose molecules due to the presence of the glucose-binding solute.

According to some embodiments, the fluid may comprise a first solute and a second solute. The first solute may be the glucose-binding solute, and the second solute may be bindable with the first solute. The first solute may for example be or comprise a lectin. Additionally or alternatively, the second solute may be or comprise a polysaccharide. Since the first solute is bindable with glucose and the second solute is bindable with the first solute, when glucose enters in contact with the fluid confined in the cavity, for example through the membrane, the second solute and the glucose molecules will compete for binding with the first solute. The first solute may have a higher affinity (bindability) with respect to glucose than the second solute.

The membrane may be permeable to a target solute, in particular to glucose. Further, the membrane may be impermeable to other substances, in particular to the previously mentioned first and second solutes that may be solved in the fluid comprised in the cavity. The first solute and the second solute may be present in the cavity as solutes solved in the fluid confined therein and may be enclosed in the cavity by the membrane.

According to a preferred embodiment, the first solute may be a lectin, the second solute may be a polysaccharide and the membrane may be permeable to glucose. At low glucose concentrations in the cavity, the presence of glucose on the external side of the membrane will give rise to a flow of glucose molecules through the membrane into the cavity, in a trend to equalize glucose concentrations on both sides of the membrane. The glucose molecules that enter the cavity through the membrane will compete with the polysaccharide molecules to bind with the lectin molecules. Since an affinity (bindability) of glucose with respect to the lectin is greater than an affinity (bindability) of the polysaccharide with respect to the lectin, as a concentration of glucose within the cavity increases, the release of free polysaccharide molecules previously bound with the lectin will trigger an increase of osmotic pressure within the cavity. Such increase in the osmotic pressure in the cavity can be sensed by the SAW sensor. The process is reversible: a decrease in glucose concentration on the external side of the membrane will cause an inverse flow of glucose molecules through the membrane from the interior of the cavity to the exterior of the cavity, thereby triggering a re-binding of the free polysaccharide molecules with the leptin molecules and hence a decrease of osmotic pressure within the cavity.

The SAW sensor of any of the embodiments of the first aspect of the invention may hence be advantageously used as an osmotic pressure sensing component in an osmotic sensor device according to the invention, which may be usable for detecting glucose levels in vivo in a patient, in particular in a patient suffering from diabetes.

In preferred embodiments of the invention, the osmotic sensor device may be configured as an implantable sensor implantable in a body of a patient. The device may be implantable in the body of the patient such that the membrane of the SAW sensor is in contact with a body fluid of the body of the patient, in particular with blood and/or with interstitial fluid of the patient.

According to preferred embodiments, the osmotic sensor device may further comprise a processing unit configured for detecting the osmotic pressure sensed by the SAW sensor, in particular based on the output electric signals generated by an output IDT of the SAW sensor. Additionally or alternatively, the processing unit may be configured for determining a concentration of a target solute in a test fluid arranged in contact with the membrane based on said osmotic pressure. The target solute may preferably be or comprise glucose. The processing unit may hence output a signal containing information about the osmotic pressure in the cavity and/or about a concentration of a solvent, for example glucose, in a test fluid arranged in contact with the membrane. The processing unit may be an external processing unit remotely arranged with respect to remaining components of the osmotic sensor device. A communication between the processing unit and the osmotic sensor device, in particular the SAW sensor thereof, may be wireless, for example by Wi-Fi, radio waves, Bluetooth or the like.

In some preferred embodiments, the osmotic sensor device may further comprise a communication unit configured for communicating with an external processing unit for communicating the osmotic pressure sensed by the SAW sensor and/or the previously mentioned concentration of a target solute. A person skilled in the art will understand that the osmotic pressure and/or the concentration of the target solute in a test fluid arranged in contact with the membrane may be transferred directly encoded in signals and/or as raw data related to the osmotic pressure and/or to the concentration of the target solute in a test fluid arranged in contact with the membrane, which may allow determining the osmotic pressure and/or the concentration of the target solute in the test fluid arranged in contact with the membrane upon further processing. The communication between the communication unit and the external processing unit may be wireless, for example by means of Wi-Fi, radio waves, Bluetooth or the like.

The SAW sensor of an osmotic sensor device according to the second aspect of the invention may be any of the SAW sensors of the embodiments of the first aspect of the invention that were previously described. In some preferred embodiments, the SAW sensor may comprise one or more resonators structures as previously defined for the SAW sensor of the first aspect of the invention. In such embodiments, at least one of the one or more resonators structures may be arranged over the cavity.

In preferred embodiments, the osmotic sensor device may further comprise a secondary cavity for confining therein a fluid between the membrane (or a further membrane) and the substrate of the SAW sensor. The secondary cavity may structurally and/or functionally identical to the previously mentioned (primary) cavity. The SAW sensor may comprise a first one of the one or more resonators structures and a second one of the one or more resonators structures, for example with an IDT device able to operate both as input IDT and as output IDT arranged between a first resonator structure and a second resonator structure. In such embodiments, the first resonator structure may be arranged over the (primary) cavity and the second resonator structure may be arranged over the secondary cavity. The input IDT and/or the output IDT may be arranged without overlapping any of the cavities and/or between the cavity and the secondary cavity. Additionally or alternatively, a first portion of the piezoelectric layer, on which the first resonator structure may be arranged, may be arranged over the (primary) cavity, while a second portion of the piezoelectric layer, on which the second resonator structure may be arranged, may be arranged over the secondary cavity, and while the input IDT and/or output IDT may be arranged on a third portion of the piezoelectric layer, disjoint from the first and second portions of the piezoelectric layer, possibly without overlapping any of the cavities and/or between the (primary) cavity and the secondary cavity.

In such embodiments, the osmotic sensor device may further comprise a processing unit configured for detecting the osmotic pressure in one or both of the (primary) cavity and the secondary cavity based on an interference between surface acoustic waves reflected by the first resonator and surface acoustic waves reflected by the second resonator. This may, in particular, be the case when an IDT device able to operate both as input IDT and as output IDT is arranged between the first resonator and the second resonator, with each of the first and second resonators and/or with each of corresponding (possibly disjoint) portions of the piezoelectric layer arranged over a corresponding one of the cavities. The cavity on which the first resonator and/or the first portion of the piezoelectric layer is arranged may be filled with the previously described fluid, which may contain the previously described first solute and the previously described second solute. The secondary cavity may be filled with another fluid or with a vacuum and may provide a reference for interferometric measurements of a concentration of a target solute in the (primary) cavity by the IDT device and/or in a test fluid arranged in contact with a corresponding portion of the membrane. The membrane closing both cavities may be the same membrane, with a first portion of the membrane closing the (primary) cavity and a second portion of the membrane closing the secondary cavity or may be different membranes. Alternatively, the (primary) cavity may be closed by the previously mentioned membrane, while the secondary cavity may be closed by an independent, possible impermeable, membrane. As previously described, the first and second resonator structures may be structurally identical to each other and/or may be symmetrically arranged with respect to the IDT device.

Notably, a detection of osmotic pressure in the cavity based on interferometric measurements may provide an increased robustness and reliability as compared to the sensing of an osmotic pressure using a conventional SAW sensor.

In preferred embodiments, the secondary cavity may be formed within the substrate of the SAW sensor. The secondary cavity may be enclosed by the membrane or the further membrane and, further, by the substrate and/or the piezoelectric layer of the SAW sensor. In embodiments in which the (primary) cavity is also formed within the substrate of the SAW sensor, with the (primary) cavity being enclosed by the membrane and by the substrate and/or by the piezoelectric layer of the SAW sensor, the cavity and the secondary cavity may be mutually separated by a portion of the substrate of the SAW sensor.

A further aspect of the invention refers to a method of manufacturing a SAW sensor, in particular a SAW sensor according to any of the embodiments of the first aspect of the invention described above. The method comprises a step of providing a substrate, in particular a substrate as described above, a step of depositing a piezoelectric layer on the substrate by means of a deposition process, the piezoelectric layer possibly corresponding to the piezoelectric of any of the previously described embodiments, and a step of arranging one or more interdigital transducers on the piezoelectric layer, the one or more IDTs possibly corresponding to the input IDT, the output IDT and/or the IDT device described above.

Preferably, the deposition process used for depositing the piezoelectric layer on the substrate may be a physical deposition process, more preferably a sputtering process. This allows forming a piezoelectric layer having a thickness much smaller than the thickness of the underlying substrate, in particular according to any of the previously described configurations with respect to thickness ratios and dimensions. Sputtering may allow manufacturing a SAW sensor according to the invention at reduced costs at wafer-level and with high manufacturing speeds. However, other deposition processes, in particular a chemical deposition process, such as atomic layer deposition (ALD), chemical vapor deposition (CVD) or a Langmuir-Blodgett deposition process may also be used.

### BRIEF DESCRIPTION OF THE DRAWINGS

FIG. 1 shows a schematic top view of a SAW sensor according to an embodiment of the invention.

FIG. 2 shows a schematic side view of the SAW sensor of FIG. 1.

FIG. 3 shows a schematic top view of a SAW sensor according to an embodiment of the invention.

FIG. 4 shows a schematic side view of the SAW sensor of FIG. 3.

FIG. 5 shows a schematic top view of a SAW sensor according to an embodiment of the invention.

FIG. 6 shows a schematic top view of the SAW sensor of FIG. 5 connected to an antenna structure according to an embodiment of the invention.

FIG. 7 shows a schematic side view of the SAW sensor of FIG. 5

FIG. 8 shows a schematic top view of a SAW sensor according to an embodiment of the invention.

FIG. 9 shows a schematic side view of the SAW sensor of FIG. 8.

FIG. 10 shows a schematic top view of a SAW sensor according to an embodiment of the invention.

FIG. 11 shows a schematic side view of the SAW sensor of FIG. 10.

FIG. 12 shows a schematic side view of an osmotic sensor device according to an embodiment of the invention.

FIG. 13 shows the schematic side view of the osmotic sensor device of FIG. 11 now including a fluid in the cavity.

FIG. 14 shows a schematic side view of an implantable osmotic sensor device according to an embodiment of the invention.

FIG. 15 shows a schematic view of an osmotic sensor device according to an embodiment of the invention.

FIG. 16 shows a schematic view of an osmotic sensor device according to another embodiment of the invention.

FIG. 17 shows a schematic side view of an osmotic sensor device according to a further embodiment of the invention.

FIG. 18 shows a schematic side view of an osmotic sensor device according to a further embodiment of the invention.

FIG. 19 shows a schematic side view of an osmotic sensor device according to a further embodiment of the invention.

FIG. 20 shows a schematic side view of an osmotic sensor device according to a further embodiment of the invention.

FIG. 21 shows a schematic side view of an osmotic sensor device according to a further embodiment of the invention.

FIG. 22 is a schematic flow diagram of a method for manufacturing a SAW sensor according to embodiments of the invention.

### DETAILED DESCRIPTION

For the purposes of promoting an understanding of the principles of the invention, reference will now be made to a preferred embodiment illustrated in the drawings, and specific language will be used to describe the same. It will nevertheless be understood that no limitation of the scope of the invention is thereby intended, such alterations and further modifications in the illustrated apparatus and such further applications of the principles of the invention as illustrated therein being contemplated as would normally occur now or in the future to one skilled in the art to which the invention relates.

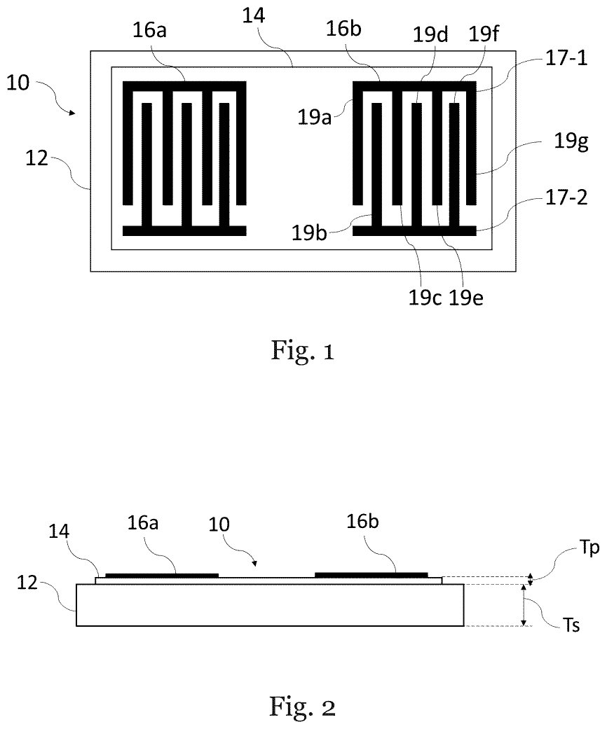

FIG. 1 shows a schematic top view of a SAW sensor 10 according to some embodiments of the invention. The sensor 10 comprises a flexible substrate 12 made of an elastic material such as silicon nitride, in particular made of or comprising Si₃N4, on which a piezoelectric layer 14 is deposited. The piezoelectric layer 14 is made of a piezoelectric material such as a AlN and can be deposited on the substrate 12 by means of a deposition process, for example by means of a sputtering process.

Therefore, as seen in the side view of FIG. 2, a thickness Tp of the piezoelectric layer 14 is considerably smaller than a thickness of the underlying substrate Ts. For example, the thickness Tp of the piezoelectric layer 14 can be 500 to 5000 times smaller than the thickness Ts of the substrate Ts. In the exemplary embodiment shown in FIGS. 1 and 2, the thickness Tp of the piezoelectric layer is 100 nm, while the thickness Ts of the substrate Ts is 500 μm. However, other absolute values of the thicknesses Tp and Ts as are possible within the context of the present invention.

As shown in FIG. 1, the substrate 12 and the piezoelectric layer deposited thereon may have a rectangular shape, although this is just an example and any other shapes are possible. In the example under consideration, the dimensions of the substrate may be 2 mm×1,5 mm and the dimensions of the piezoelectric layer may be 1,5×0,75 mm.

As seen in FIGS. 1 and 2, an input IDT 16a and an output IDT 16b are both arranged on the piezoelectric layer 14 and spaced apart in the side view shown in FIG. 2. Each of the IDTs 16a and 16b comprises a respective first conductive element 17-1 and a second conductive element 17-2, with interlocked fingers 19a-19g. The first conductive element 17-1 comprises fingers 19a, 19c, 19e and 19g joint by a common transverse portion and the second conductive element 17-2 comprises fingers 19b, 19d and 19f, joint by a common transverse portion, and interdigitized (interlocked) with the fingers 19a, 19c, 19e and 19g of the first conductive element 17-1. The number and specific shape of the fingers is exemplary.

The input IDT 16a may be used for transforming electric signals into surface acoustic waves via the inverse piezoelectric effect. The surface acoustic waves generated by the input IDT 16a propagate along the piezoelectric layer 14 and possibly along the substrate 12 (the surface acoustic waves may penetrate into the substrate 12) to the output IDT 16b, where the sound surface waves are received and transformed, by means of the piezoelectric effect, into output electric signals.

Due to the piezoelectric nature of the piezoelectric layer 14, the physical conditions of the propagation of the surface acoustic waves along the piezoelectric layer 14 between the input IDT 16a and the output IDT 16b are affected by mechanical conditions to which the piezoelectric layer 14 may be subject. Therefore, the output electric signals may encode information about said mechanical conditions, for example about a pressure, to which the piezoelectric layer and/or the substrate may be exposed. Consequently, information about said mechanical conditions may be obtained by processing and analyzing the output electric signals outputted by the output IDT.

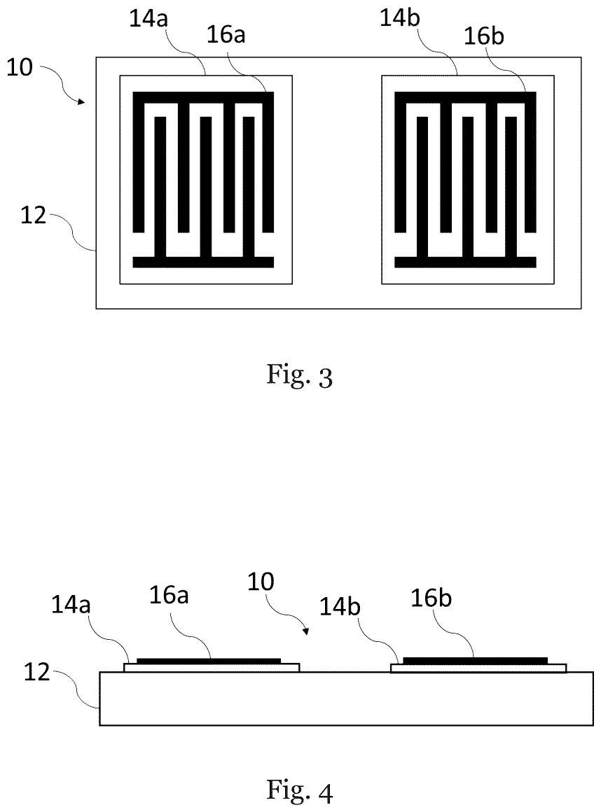

The information provided above with respect to the structure, dimensions, thicknesses and ratios of the substrate 12 and the piezoelectric layer 14 of the SAW sensor 10 shown in FIGS. 1 and 2 may apply in general to any SAW sensor according to the present invention, and in particular to the embodiments to be described below with respect to FIGS. 3 to 11 but will not be repeated each time on behalf of brevity. For the same reason, whenever identical reference signs are used in the figures, it should be understood, unless indicated otherwise, that corresponding or identical elements of a SAW sensor are being referred to.

FIG. 3 shows a schematic top view, corresponding to the view of FIG. 1, of a SAW sensor 10 according to another embodiment of the invention. FIG. 4 shows a schematic side view of the SAW sensor 10 of FIG. 3, corresponding to the side view of FIG. 2. One difference between the SAW sensor 10 shown in FIGS. 3 and 4 with respect to the SAW sensor are shown in FIGS. 1 and 2 is that, in the SAW sensor 10 shown in FIGS. 3 and 4, the input IDT 16a is arranged on a first portion 14a of the piezoelectric layer whereas the output IDT 16b is arranged on a second portion 14b of the piezoelectric layer. The first and second portions 14A, 14b of the piezoelectric layer are mutually separated by the substrate 12. In a manufacturing phase, the portions 14a and 14b of the piezoelectric layer may be deposited during one same deposition process or in separated deposition processes. For example, the portions 14a and 14b may be formed by first depositing a single piezoelectric layer and thereupon patterning the piezoelectric layer, for example using an etching technique or a photolithographic technique, to form the separate portions 14a and 14b.

As compared to the embodiment shown in FIGS. 1 and 2, the configuration of the piezoelectric 14 divided in the first and second portions 14a and 14b, respectively receiving the input IDT 16a and the output IDT 16b, may allow manufacturing a SAW sensor 10 according to the present invention with a reduced amount of piezoelectric material for forming the piezoelectric layer 14. This notwithstanding, the working principle of the SAW sensor 10 of the exemplary embodiment shown in FIGS. 3 and 4 may correspond to the working principle of the SAW sensor of FIGS. 1 and 2. An input electric signal provided to the input IDT 16a is transformed, due to the inverse piezoelectric effect taking place on the first portion 14a of the piezoelectric layer, into surface acoustic waves that propagate along the first portion 14a, the substrate 12 and the second portion 14b to the output IDT 16b. Upon being received by the output IDT 16b, the surface acoustic waves received from the input IDT 16a are converted into electrical signals due to the piezoelectric effect on the second portion 14b of the piezoelectric layer 14.

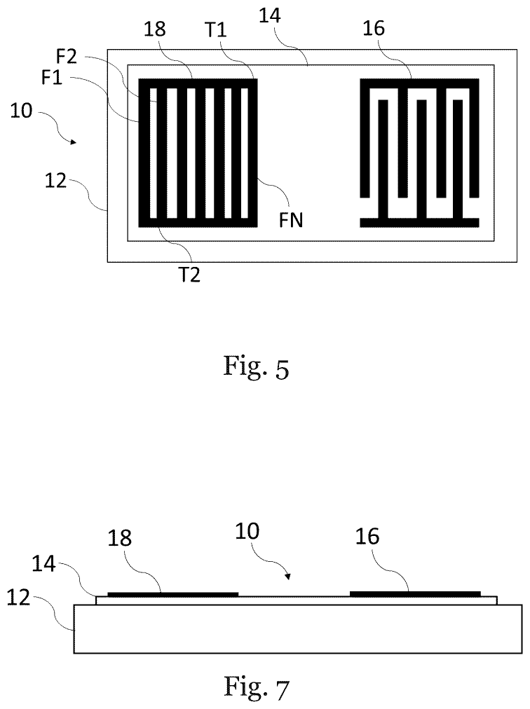

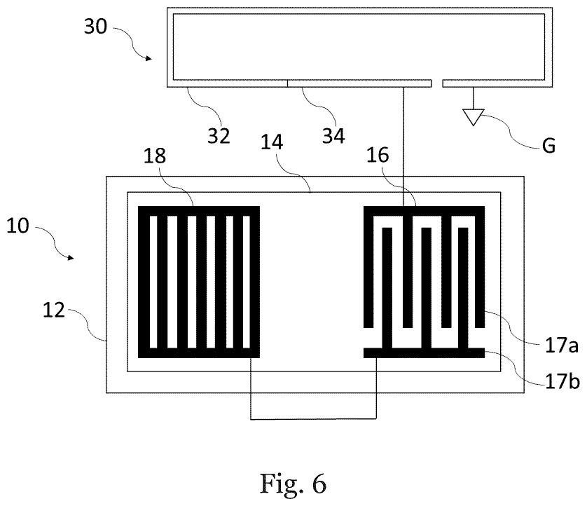

FIGS. 5 to 7 shows a schematic view of a SAW sensor according to a further embodiment of the invention. In the exemplary embodiments previously described with respect to FIGS. 1 to 4, the input IDT 16a and the output IDT 16b are configured as separate devices. In the exemplary embodiment of FIGS. 5 to 7, one and the same IDT device 16 is able to operate both as input IDT and as output IDT. As seen in the schematic top views of FIGS. 5 and 6 and in the schematic side view of FIG. 7, the SAW sensor 10 according to this exemplary embodiment further comprises a resonator structure 18 that is arranged on the piezoelectric layer 14, spaced apart from the IDT device 16. The resonator structure 18 is configured as an electrode lattice structure and is configured for reflecting surface acoustic waves. The resonator structure 18 may be a floating structure not being electrically coupled with any other structure.

As exemplary seen in FIG. 5, the resonator structure 18 can comprise a plurality of finger portions F1 F2, . . . , FN extending in parallel between first and second transverse portions T1 and T2. The finger portions F1, F2, . . . , FN are preferably arranged at a regular mutual distance between adjacent finger portions corresponding to a separation distance between adjacent fingers 19a-19g of the IDT device (see FIG. 1, showing fingers 19a-19g described above). This can ensure fulfilment of the Bragg condition.

However, other non-floating configurations of the resonator structure 18 are possible. For example, as shown in FIG. 6, the two conductive elements 17a and 17b of the IDT device 16, which are interlocked (and as explained above in detail for FIG. 1), wherein one of the conductive elements 17ba can share an electrical potential with the resonator structure 18, whereas the other conductive element 17a can be connected to the antenna structure 30. The antenna structure 30, which is configured to operate in a frequency range from 2.4 GHz (ISM band) to 5 GHz, is a loop antenna comprising a loop element 32 and a coupling feed 34. The loop element 32 is formed as a wire loop with a length of 125 mm and a thickness of 1 mm and is connected to ground G. The coupling feed 34 is connected to the conductive element 17b.

Input electric signals provided to the IDT device 16 of FIGS. 5 to 7 are transformed, due to the inverse piezoelectric effect, into surface acoustic waves. Here, the IDT device 16 acts as input IDT. The surface acoustic waves propagate along the piezoelectric layer 14 and possibly along the substrate 12 to the resonator structure 18, where they are reflected back to the IDT device 16. The structure of the IDT device 16, for example the distance between adjacent finger elements 19a-19d, as well as a distance from the IDT device 16 and the resonator structure 18 are hence adapted for this purpose in a manner that is accessible to the skilled person. When the surface acoustic waves reflected by the resonator 18 reach the IDT device 16, the IDT device 16 now operates as output IDT and transforms the reflected surface acoustic waves into electric signals due to the piezoelectric effect. As in the case of the exemplary embodiment described above with respect to FIGS. 3 and 4, although the resonator 18 and the IDT device 16 shown in FIGS. 5 to 7 are arranged on the same portion of the piezoelectric layer 14, in other related embodiments, the resonator 18 and the IDT device 16 can be arranged on different and separated portions of the piezoelectric layer 14 (cf. portions 14a and 14b shown in FIGS. 3 and 4).

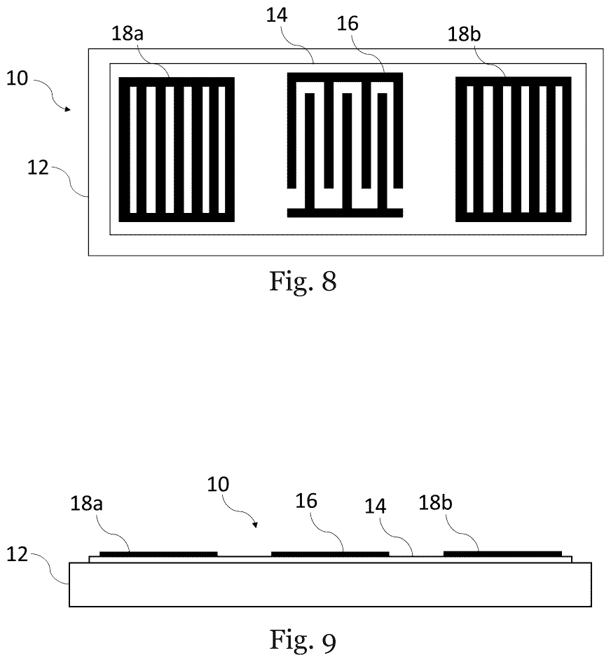

FIGS. 8 and 9 show an exemplary embodiment of a SAW sensor 10 according to further embodiments of the invention. FIG. 8 shows a schematic top view, whereas FIG. 9 shows a schematic side view of the SAW sensor 10. In this exemplary embodiment, as compared to the exemplary embodiment described with respect to FIGS. 5 to 7, two resonator structures 18a and 18b are arranged on the piezoelectric layer 14, with the IDT device 16 being arranged between the first resonator 18a and the second resonator 18b. The first resonator 18a and the second resonator 18b are identical lattice structures and are symmetrically arranged with respect to the IDT device 16. The first resonator 18a and the second resonator 18b can have the structure previously described for the resonator structure 18 of FIG. 5.

When input electric signals are provided to the IDT device 16 of FIGS. 8 and 9, the IDT device 16 acts as input IDT transforming the input electric signals, via the inverse piezoelectric effect, into surface acoustic waves that propagate along piezoelectric layer 14 and possibly along the substrate 12 from the IDT device 16 to each of the first and second resonator structures 18a and 18b, at which the surface acoustic waves are reflected back to the IDT device 16. When the reflected surface acoustic waves, coming from each of the first and second resonator structures 18a and 18b reach the IDT device 16, the IDT device 16, now acting as output IDT, converts the incoming surface acoustic waves, via the piezoelectric effect, into output electric signals. Due to the IDT device 16 being arranged between the first and second resonator structures 18a and 18b, the IDT device 16 is configured for generating the output electric signals based on an interference of the surface acoustic waves reflected at each of the resonator structures 18a and 18b.

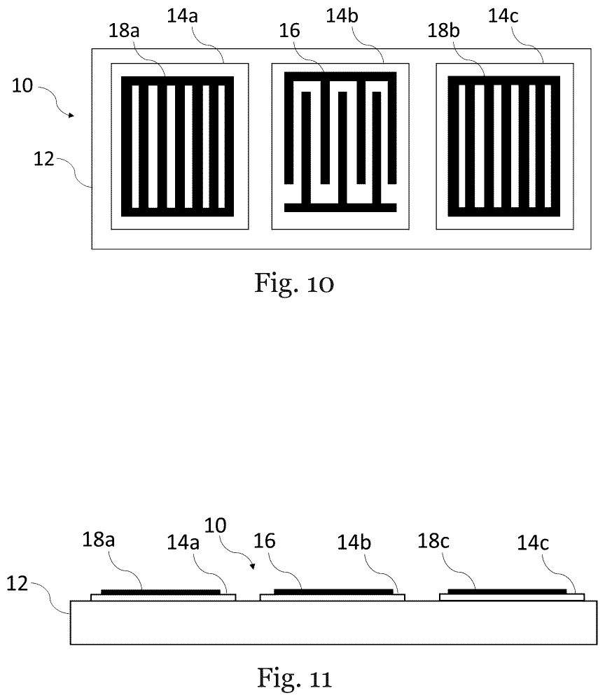

In the exemplary embodiment shown in FIGS. 8 and 9, the resonator structures 18a and 18b and the IDT device 16 are all arranged on a same portion of the piezoelectric layer 14. In contrast, FIGS. 10 and 11 respectively show a schematic top view and a schematic side view of a related embodiment of the SAW sensor 10 according to the invention in which the resonator structures 18a and 18b and the IDT device 16 are each arranged on a respective portion 14a, 14b and 14c of the piezoelectric layer. The different portions 14a, 14b and 14c of the piezoelectric layer 14 are mutually separated, in particular by portions of the substrate 12 extending therebetween. During the manufacturing process of the SAW sensor 10, the portions 14a, 14b and 14c may be deposited on the substrate 12 during the same deposition process or in separate deposition processes. For example, the portions 14a, 14b and 14c may be formed as disjoint portions by first depositing a single piezoelectric layer and thereupon patterning the piezoelectric layer, for example using an etching technique or a photolithographic technique, to form the separate portions 14a, 14b and 14c.

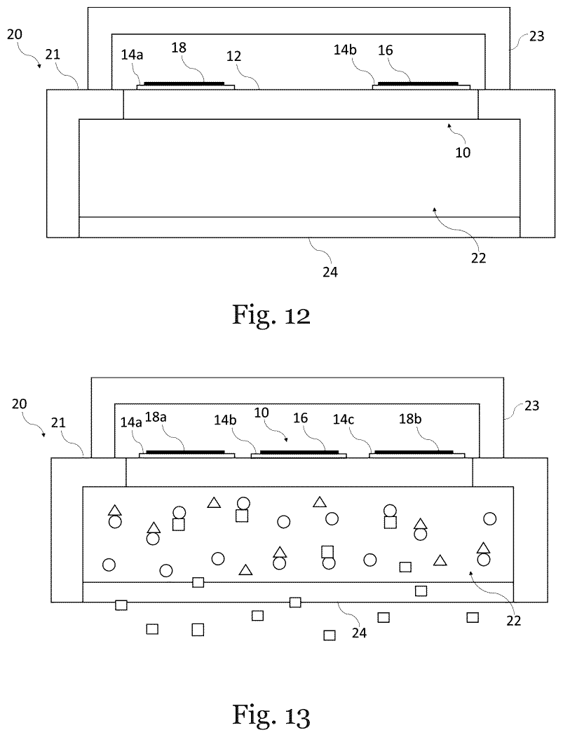

FIG. 12 shows a schematic side view of an osmotic sensor device 20 according to an exemplary embodiment of the invention. The device 20 comprises a structural frame 21 in which a cavity 22 is formed. The cavity is defined by the structure frame 21 and by a membrane 24 that enclose the cavity 22, such that a fluid can be confined within the cavity 22. The membrane is configured for allowing osmotic diffusion into and/or from the cavity 22. The device 20 further comprises a SAW sensor 10 integrated therein, such that the substrate 12 of the SAW sensor 10 is in contact with the cavity 22. Therefore, the substrate 12 is exposed to pressure variations within the cavity 22. Thus, when a fluid received within the cavity 22 undergoes pressure variations, in particular variations in osmotic pressure, such pressure variations will be sensed by the SAW sensor 10 and will manifest in the output electric signals outputted by the IDT device 16 or a corresponding output IDT 16b of the SAW sensor 10.

Notably, the exemplary embodiment shown in FIG. 12 includes a SAW sensor 10 corresponding to the exemplary embodiment described above with respect to FIGS. 5 to 7. However, the SAW sensor 10 integrated in the osmotic sensor device 20 may correspond to any sensor device 10 according to embodiments of the present invention, and in particular according to any of the exemplary embodiments described above with respect to FIGS. 1 to 11.

Also shown in FIG. 12 is a cover 23 that covers and overlies the SAW sensor 10 on a side of the SAW sensor opposite the cavity 22. A space between the cover 23 and the SAW sensor may be filled with a gas, for example with N₂, or with vacuum. The cover provides protection to the components of the SAW sensor 10 that would otherwise be exposed to the environment, i.e., to the piezoelectric layer portions 14a and 14b, to the IDT device 16 and to the resonator structure 18.

FIG. 13 shows a further schematic view of the osmotic sensor device 20 of FIG. 12, in which the cavity 22 is filled with a liquid that is a solution comprising a first glucose-binding solute, indicated with circular markings in FIG. 13, such as for example a lectin, and further comprising a second solute that is bindable with the first solute. The second solute may for example be a polysaccharide and is indicated in FIG. 13 with triangular markings. The membrane 24 is selectively permeable to a target substance, for example glucose, which is indicated in FIG. 13 with square markings. The membrane 24 is however impermeable to the first solute and the second solute contained in the fluid confined within the cavity 22, in the present example the lectin (circular markings) and the polysaccharide (triangular markings).

In the exemplary embodiment of FIG. 13, glucose molecules may diffuse through the membrane 24 due to osmotic diffusion when the membrane 24 is exposed to a test fluid having a different glucose concentration than the fluid that is confined within the cavity 22. Such test fluid may for example be blood or interstitial fluid of a patient suffering from diabetes. The polysaccharide (triangular markings) and the glucose (square markings) will hence compete for binding with the lectin (circular markings). When the test fluid has a higher glucose concentration than the fluid that is confined within the cavity 22, glucose molecules will flow from the exterior of the cavity 22 into the cavity 22 across the membrane 24 and bind with the lectin, thereby causing the lectin to dissociate from the polysaccharide. The resulting free polysaccharide molecules will trigger an increase in osmotic pressure within the cavity 22 that will be sensed by the SAW sensor 10. Conversely, when a concentration of glucose in the fluid that is confined within the cavity 22 is greater than a concentration of glucose in the test fluid, glucose molecules will flow from the cavity 22 to the exterior across the membrane 24, triggering the polysaccharide molecules to re-bind with the lectin, thereby triggering a decrease in osmotic pressure within the cavity 22 that will be sensed by the SAW sensor 10.

In either case, by processing the electric output signals outputted by the SAW sensor 10, it is possible to infer the osmotic pressure in the cavity 22 and/or the concentration of glucose in the test fluid.

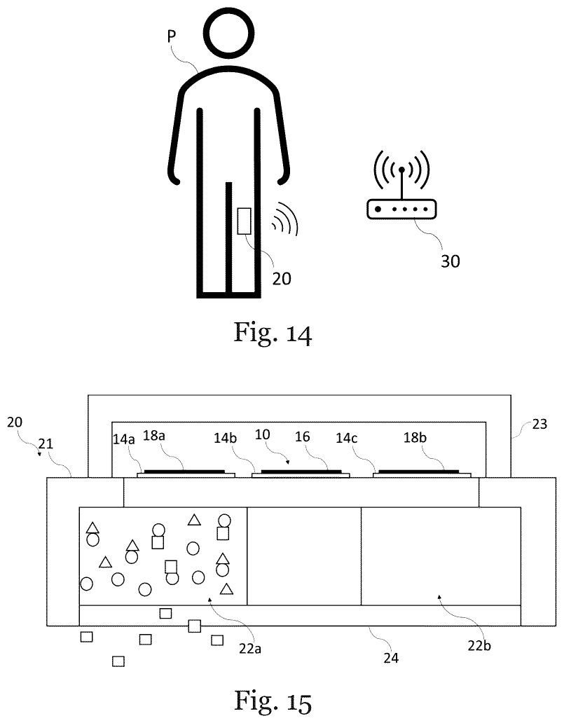

As illustrated in FIG. 14, an osmotic pressure sensor device 20 according to embodiments of the present invention can be used as an implantable sensor implantable in a body of a patient P. If the implantable sensor 20 is implanted in the body of the patient such that the membrane 24 is in contact with a body fluid of the patient, such as for example with blood or with interstitial fluid of the patient P, the implantable sensor 20 can for example be used for detecting a level of glucose in the blood and or the interstitial fluid of the patient. This may be useful for example for treating patients suffering from diabetes. The cover 23 protects the body of the patient from entering into direct contact with the SAW sensor 10.

As shown in FIG. 14, the sensor device 20 may further comprise a processing unit 30 that may be independent from the rest of the components of the sensor device 20 that are implanted within the body of the patient P. The processing unit 30 is configured for wirelessly communicating with the implanted sensor device 20, for example for receiving output electromagnetic waves encoding the electric output signals outputted by the output IDT or by the IDT device of the SAW sensor 10 and is configured for determining the osmotic pressure sensed by the SAW sensor 10, i.e. the osmotic pressure in the cavity 22, based on those output electric signals. Based on that, the processing unit 30 may determine a concentration of a target solute in the blood or the interstitial fluid of the patient, for example a glucose concentration.

In order to communicate with the processing unit 30, the sensor device 20, in particular the SAW sensor 10, may comprise a communication unit (not shown) for communicating with the external processing unit 30, in particular wirelessly. The communication unit, which may for example comprise one or more antennas can be configured for communicating to the external processing unit 30 the osmotic pressure sensed by the SAW sensor 10 and/or the concentration of the target solute in the blood or interstitial fluid of the patient, which may previously have been determined by an internal processing unit of the sensor device 20. Alternatively, the communication unit can be configured for transferring to the processing unit 30 all raw data obtained by the SAW sensor as electric output signals and the processing unit 30 may be configured for determining the osmotic pressure in the cavity 22 and/or the concentration of the target solute in the blood or interstitial fluid of the patient based thereon.

FIG. 15 shows a schematic side view of a related embodiment of an osmotic sensor device 20 according to the present invention, in which the SAW sensor device 10 integrated in the sensor device 20 corresponds to a SAW sensor 10 as described with respect to the exemplary embodiment shown in FIGS. 8 and 9 or in FIGS. 10 and 11. The SAW sensor 10 comprises an IDT device 16, which is able to act both as input IDT and as output IDT, which is arranged between a first resonator structure 18a and a second resonator structure 18b. The device 20 comprises, apart from the cavity 22a, which corresponds to the cavity 22 described above with respect to the exemplary embodiment shown in FIGS. 12 and 13, a secondary cavity 22b, which is structurally and functionally identical to the primary cavity 22a. Both cavities 22a and 22b are enclosed by the structure frame 21 and by the membrane 24 and are configured for receiving and confining therein a corresponding fluid, possibly the same fluid. The first resonator structure 18a is arranged on a first portion 14a of the piezoelectric layer over the primary cavity 22a, and the second resonator structure 18b is arranged on a third portion 14c of the piezoelectric layer over the secondary cavity 22b. According to this embodiment, the measurement process described above with respect to FIG. 13 may be performed using one of the cavities only, for example using the primary cavity 22a, while the secondary cavity 22b may be left empty, containing a vacuum, or containing a reference fluid. The reference fluid can be the same solution initially contained in the primary cavity 22a. This configuration may allow a more robust and reliable determination of the concentration of a target solute in the primary cavity 22a, for example glucose, based on the use of the secondary cavity 22b as a reference and on the use of interferometry by the IDT device 16 of the SAW sensor 10, where surface acoustic waves reflected at each of the resonator structures 18a and 18b interfere with each other and the output electric signals can be generated based on such interference.

For such embodiments, the previously mentioned processing unit 30, for example the external processing unit shown in FIG. 14, can be configured for determining the osmotic pressure in the cavity 22a or a corresponding concentration of a target solute (see square markings in FIG. 15) based on the interference between surface acoustic waves reflected by the first resonator structure 18a and by the second resonator structures 18b, as previously explained.

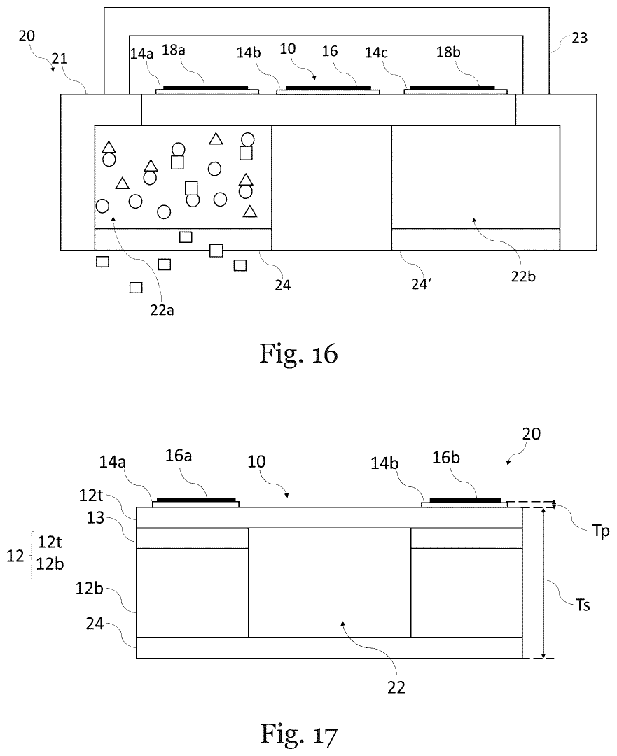

FIG. 16 shows a schematic side view of a variation of the osmotic sensor device 20 of FIG. 15, in which the secondary cavity 22b is enclosed by an impermeable membrane 24′ that is independent of the membrane 24 that encloses the primary cavity 22a. The cavities 22a and 22b are mutually separated by a portion of the structural frame 21.

FIGS. 17 to 21 show osmotic sensor devices according to further embodiments of the invention, in which the cavity 22 is formed within the substrate 12 and is enclosed by the membrane 24, the substrate 12 and/or the piezoelectric layer 14 of the SAW sensor 10.

FIG. 17 shows a schematic side view of an osmotic sensor device 20 according to a further embodiment of the invention. As compared to the osmotic sensor device of the embodiment shown in FIG. 12, in the osmotic sensor device 20 according to the embodiment of FIG. 17, the cavity 22 is formed within the substrate 12, rather than within a frame structure 21. The cavity 22 is enclosed by the substrate 12 and by the membrane 14. A top portion 12t of the substrate 12 extends over the cavity 22 and encloses the cavity 22 from above and a bottom portion 12b of the substrate 12 encloses the cavity 22 laterally. The membrane 24, which corresponds to a membrane 24 as described above for FIGS. 12 to 16, encloses the cavity 22 from below. First and second portions 14a and 14b of the piezoelectric layer are formed on the top portion 12p of the substrate 12. An input IDT 16a is formed on the first portion 14a of the piezoelectric layer and an output IDT 16b is formed on the second portion 14b. Thus, the structure of the piezoelectric layer and the IDT devices of the SAW sensor 10 integrated in this embodiment of the osmotic sensor device 20 corresponds to the structure explained above with respect to FIGS. 3 and 4. However, the SAW sensor 10 integrated in the osmotic sensor device 20 of FIG. 17 may correspond to any sensor device 10 according to embodiments of the present invention, and in particular according to any of the exemplary embodiments described above with respect to FIGS. 1 to 11.

In the exemplary embodiment shown in FIG. 17, the substrate 12 further comprises an etch stop layer 13 made of SiO₂ arranged between the bottom portion 12b of the substrate 12 and the top portion 12t. The cavity 22 is also laterally enclosed by the etch stop layer 13. The substrate 12, including the top portion 12t, is made of silicon with (111) crystallographic orientation.

In the exemplary embodiment shown in FIG. 17, the substrate 12, including the top portion 12t, the etch stop layer 13 and the bottom portion 12b, has a thickness corresponding to the thickness Ts explained above with respect to FIG. 2, while the piezoelectric layer (including portions 14a and 14b) has a thickness corresponding to the thickness Tp explained above with respect to FIG. 2. A thickness of the top portion 12t of the substrate may be smaller than a thickness of the piezoelectric layer. For example, the piezoelectric layer can have a thickness from 200 nm to 1 μm, the top portion 12t of the substrate 12 can have a thickness from 500 nm to 2 μm, the etch stop layer 13 can have a thickness from 100 nm to 1 μm, and the bottom portion 12b of the substrate 12 can have a thickness from 200 μm to 500 μm.

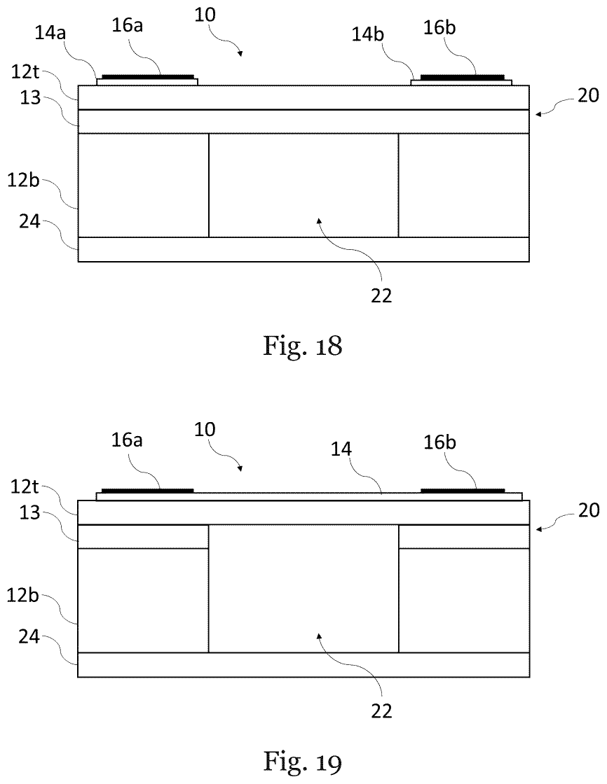

FIG. 18 shows a variation of the embodiment shown in FIG. 17, in which both the etch stop layer 13 and the top portion 12t of the substrate 12 extend over the cavity 22. The cavity 22 is laterally enclosed by the bottom portion 12b of the substrate 12, enclosed from above by the etch stop layer 13 and by the top portion 12t of the substrate 12, and enclosed from below by the membrane 24.

FIG. 19 shows a variation of the embodiments shown in FIGS. 17 and 18, in which both the piezoelectric layer 14 (now with a single portion on which both IDT devices 16a and 16b are formed) and the top portion 12t of the substrate 12 extend over the cavity 22. In this exemplary embodiment, the etch stop layer 13 does not extend over the cavity 22. The cavity 22 is hence laterally enclosed by the bottom portion 12b of the substrate 12 and by the etch stop layer 13, enclosed from above by the top portion 12t of the substrate 12 and by the piezoelectric layer 14, and enclosed from below by the membrane 24.

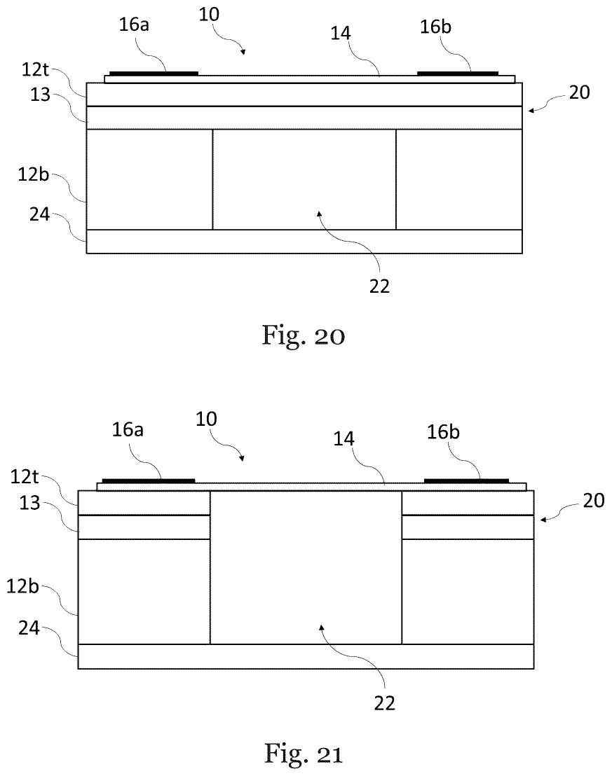

FIG. 20 shows a variation of the embodiments shown in FIGS. 17 to 19, in which the piezoelectric layer 14 (now with a single portion on which both IDT devices 16a and 16b are formed), the etch stop layer 13 and the top portion 12t of the substrate 12 extend over the cavity 22. The cavity 22 is hence laterally enclosed by the bottom portion 12b of the substrate 12, enclosed from above by the etch stop layer 13, the top portion 12t of the substrate 12 and by the piezoelectric layer 14, and enclosed from below by the membrane 24.

FIG. 21 shows a variation of the embodiments shown in FIGS. 17 to 20, in which the piezoelectric layer 14 (now with a single portion on which both IDT devices 16a and 16b are formed) extends over the cavity 22. In this exemplary embodiment, the etch stop layer 13 and the top portion 12p of the substrate 12 do not extend over the cavity 22. The cavity 22 is hence laterally enclosed by the substrate 12 and the etch stop layer 13, enclosed from above by the piezoelectric layer 14 and enclosed from below by the membrane 24.

In the exemplary embodiments shown in FIGS. 17 to 21, the osmotic sensor device 20 needs not comprise a frame structure like the frame structure 21 described for the embodiments of FIGS. 12, 13 and 15. However, despite not being shown in FIGS. 17 to 21, the osmotic sensor device 20 can comprise, also in the exemplary embodiments shown in these figures, a cover 23 like the cover previously described for the embodiments of FIGS. 12, 13 and 15, extending over the top surface of the SAW sensor 10, for example over the piezoelectric layer 14 and over the IDT devices 16a, 16b.

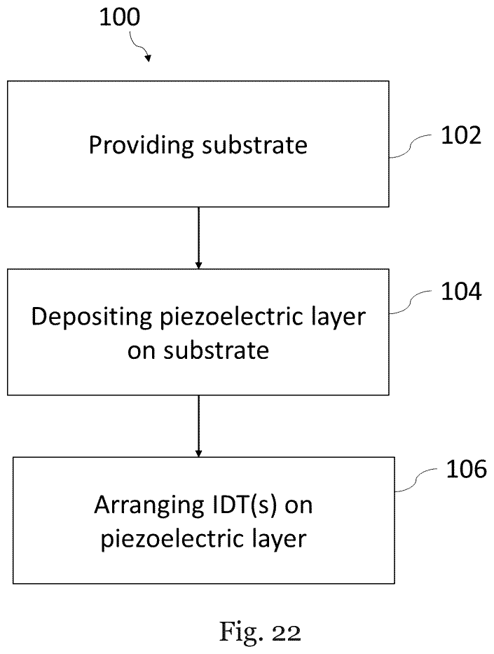

FIG. 22 is a schematic flow diagram illustrating a method 100 for manufacturing a SAW sensor according to embodiments of the present invention. The method 100 allows manufacturing a SAW sensor 10 according to any of the previously described embodiments of the invention, in particular the SAW sensor device 10 according to any of the exemplary embodiments described with respect to FIGS. 1 to 21.

The method 100 comprises a step 102 in which the substrate 12 is provided. The substrate may be provided as a Si-wafer, or as an SOI-(silicon on insulator)-wafer. The substrate can comprise a SiN layer on a substrate thereof.

Thereupon, at step 104, the piezoelectric layer 14 is deposited on the substrate 12. Depositing the piezoelectric layer 14 on the substrate 12 comprises a deposition process, which can for example be a physical deposition process, preferably a sputtering process. However, other deposition techniques are possible, for example chemical deposition process, such as atomic layer deposition (ALD), chemical vapor deposition (CVD) or a Langmuir-Blodgett deposition process. After depositing, the piezoelectric layer 14 may be patterned, for example using a lithographic patterning process.

After the piezoelectric layer 14 is deposited on the substrate 12 at 104, the method 100 proceeds to step 106, in which one or more IDTs, for example the input IDT 16a, the output IDT 16b and/or the IDT device 16, are arranged on the piezoelectric layer 14. The one or more IDTs can be formed on the piezoelectric layer using a patterning process, such as a lithographic patterning process.

After step 106, the resulting SAW sensor 10 can be arranged in a frame structure 21 to form an osmotic sensor device 20, for example an osmotic sensor device 20 according to any of the embodiments previously described with respect to FIGS. 12 to 15. Additionally or alternatively, a cavity 22 may be formed in the substrate 12 using an etching process, by etching the substrate from a surface thereof opposite to the top surface on which the piezoelectric layer 14 is formed. In such cases, the etch stop layer 13, if present, can prevent over-etching. After forming the cavity 22, the cavity 22 can be filled as appropriate, for example with a liquid solution comprising a lectin and a polysaccharide. After filling, the cavity 22 can be sealed with a glucose-permeable membrane 24. Finally, a cover 23 (see FIGS. 12, 13 and 15) can be arranged to cover the surface of the SAW sensor 10 on which the piezoelectric layer 14 and the IDT(s) 16 are formed. A gap between the cover 23 and the SAW sensor can be filled with vacuum or with a gas, such as N₂.

Although preferred exemplary embodiments are shown and specified in detail in the drawings and the preceding specification, these should be viewed as purely exemplary and not as limiting the invention. It is noted in this regard that only the preferred exemplary embodiments are shown and specified, and all variations and modifications should be protected that presently or in the future lie within the scope of protection of the invention as defined in the claims.

What is claimed is:

1-29. (canceled)

30. A surface acoustic wave, SAW, sensor comprising:
a substrate,
a piezoelectric layer deposited on the substrate,
an input interdigital transducer, IDT, configured for converting input electric signals into surface acoustic waves, and an output IDT configured for converting said surface acoustic waves into output electric signals, wherein the input IDT and the output IDT are arranged on the piezoelectric layer.

31. The SAW sensor of claim 30, wherein a thickness of the piezoelectric layer is 100 to 10000 times smaller than a thickness of the substrate.

32. The SAW sensor of claim 30, wherein the piezoelectric layer comprises or is made of AlN, GaN, InN, ZnO, quarz, LiNbO₃, a piezoelectric polymer or any combination thereof.

33. The SAW sensor of claim 30, wherein the substrate comprises or is made of a flexible or elastic material, preferably Si, SiN, SiOx or a combination thereof.

34. The SAW sensor of claim 30, wherein the input IDT and the output IDT are the same IDT device.

35. The SAW sensor of claim 30, further comprising one or more resonator structures configured for reflecting said surface acoustic waves to the output IDT, wherein the one or more resonator structures are arranged on the piezoelectric layer.

36. The SAW sensor of claim 30, wherein the output IDT or the input IDT is arranged on a first portion of the piezoelectric layer, wherein at least one of the one or more resonator structures is respectively arranged on further portions of the piezoelectric layer, said further portions of the piezoelectric layer being separated from said first portion of the piezoelectric layer by the substrate.

39-51. (canceled)

52. An osmotic sensor device for measuring a property of a body fluid, the sensor device comprising:
a cavity for confining therein a fluid;
a membrane for allowing osmotic diffusion into and/or from the cavity; and
a SAW sensor configured for sensing an osmotic pressure in the cavity, the SAW sensor comprising:
a substrate;
a piezoelectric layer deposited on the substrate;
an input interdigital transducer, IDT, configured for converting input electric signals into surface acoustic waves, and an output IDT configured for converting said surface acoustic waves into output electric signals, wherein the input IDT and the output IDT are arranged on the piezoelectric layer.

53. The device of claim 52, wherein the cavity is formed within the substrate of the SAW sensor, wherein the cavity is enclosed by the membrane and by the substrate or the piezoelectric layer of the SAW sensor.

54. The device of claim 52, wherein the substrate of the SAW sensor is in contact with the cavity.

55. The device of claim 52, further comprising a fluid confined in the cavity, wherein the fluid comprises a glucose-binding solute.

56. The device of claim 55, wherein the fluid comprises a first solute and a second solute, wherein the first solute is the glucose-binding solute and wherein the second solute is bindable with the first solute.

57. The device of claim 56, wherein the first solute is or comprises a lectin.

58. The device of claim 56, wherein the second solute is or comprises a polysaccharide.

59. The device of claim 55, wherein the membrane is permeable to glucose.

60. The device of claim 52, further comprising a communication unit configured for communicating with an external processing unit for communicating said osmotic pressure and/or a concentration of a target solute in a test fluid arranged in contact with the membrane, preferably wirelessly.

61. The device of claim 52, wherein the device is configured as an implantable sensor implantable in a body of a patient, such that the membrane is in contact with a body fluid of said body of a patient, in particular with blood and/or with interstitial fluid of the patient.

62. The device of claim 52, further comprising a processing unit configured for detecting an osmotic pressure sensed by the SAW sensor based on the output electric signals and/or a concentration of a target solute in a test fluid arranged in contact with the membrane based on said osmotic pressure.

63. The device of claim 62, wherein the target solute is or comprises glucose.

64. A method of manufacturing a SAW sensor, the method comprising:
providing a substrate;
depositing a piezoelectric layer on the substrate by means of a deposition process; and
arranging one or more interdigital transducers, IDT, on the piezoelectric layer, wherein the deposition process is a physical deposition process.
